(19) United States
(12) Patent Application Publication
(10) Pub. No.: US 2020/0237501 A1
(43) Pub. Date: Jul. 30, 2020

(54) INTRAOCULAR LENS

(71) Applicant: RAYNER INTRAOCULAR LENSES LIMITED, WORTHING WEST SUSSEX (GB)

(72) Inventor: PABLO BENITO LOPEZ, WORTHING WEST SUSSEX (GB)

(21) Appl. No.: 16/634,972

(22) PCT Filed: Aug. 3, 2018
(86) PCT No.: PCT/GB2018/052227

§ 371 Date: Jan. 29, 2020

(30) Foreign Application Priority Data
Aug. 4, 2017 (GB) 1712599.8

(51) Int. Cl. A61F 2/16

(52) U.S. Cl. CPC A61F 2/1601; A61F 2002/1681

(57) ABSTRACT

A deformable intraocular lens (IOL) comprises a deformable optic, having an annular edge around its periphery, and at least two haptics, each having a proximal portion and a distal portion relative to the optic. Each haptic extends from a different position on the annular edge of the optic and can be compressed. The IOL further comprises at least one tab extending from the annular edge of the optic at a position different from the proximal portion of each of the haptics. The or each tab can adjust the folding characteristics of the IOL, which in turn can control the speed and orientation of the IOL on injection into a patient's eye and ensure greater reliability of desired injection.

### FIELD OF THE INVENTION

The present invention relates to the design of an intraocular lens (IOL), and in particular an IOL for injecting into an eye of a patient using an injector device.

### BACKGROUND OF THE INVENTION

Intraocular lenses (IOLs) are now widely used in the treatment of ophthalmic conditions, both to replace the natural lens of the subject or as a secondary lens to supplement the natural lens or primary replacement IOL. One of the operative treatments used to treat cataract is that of removing a natural crystalline lens from an eye of a patient and then injecting an intraocular lens (IOL) in place of the natural crystalline lens.

IOLs typically comprise a lens portion and a pair of resilient haptics extending outwardly from opposite sides of the periphery of the lens portion. The haptics aid in locating the IOL in a correct position in the eye and in maintaining the IOL in that correct position. Such IOLs have steadily become more sophisticated with improved designs for the IOL haptics and for the annular rims of the IOL optic to ensure accurate and stable location within the lens cavity of the eye.

The majority of first generation IOLs were manufactured from rigid PMMA and were implanted into the eye using forceps through large (5-6 mm) incisions. The large incision size increased the risk of infection and could lead to induced changes in the shape of the cornea and also potentially cause astigmatism of the eye after the operation.

To prevent such disadvantages, a next generation of foldable IOLs was developed that could be introduced into the eye through a reduced incision size (2-4 mm) using an injector. Such IOLs can be employed for micro-incision cataract surgery (MICS) with a suitable injection technique.

The procedure to inject an IOL usually consists of first making an incision in the eye, then fragmenting and aspirating a clouded natural crystalline lens through the incision, and finally injecting the IOL into the eye through the incision to implant it in place of the natural crystalline lens.

It is essential for IOLs to be stored unstressed so as not to become permanently deformed over time. Accordingly, IOLs are not held in a folded condition over a long period of time, for example in storage.

More recent, disposable, single-use type injectors come preloaded with an IOL. Preloaded injectors designed for delivery of hydrophobic IOLs usually incorporate lens storage within the main injector body. Due to its simplicity, this is a more preferable option for a preloaded injector and is possible because hydrophobic IOLs can be stored in a non-hydrated or ‘dry’ state.

In some injectors, the cartridge is manipulated prior to insertion into the injector in order to fold the IOL within. For example, one format includes a pair of hinged flaps that, in a first configuration, define a chamber that holds the IOL in an unfolded state. When the flaps are hinged together, the chamber becomes reduced in size, thus folding the IOL within. This is also the case with integral cartridges, where the IOL is inserted into a loading bay of the injector (defined within the integral cartridge) at the point of use in an unfolded state and the flaps of the cartridge are then closed to fold the IOL.

IOLs injected through an injector nozzle for MICS need to emerge into the capsular bag with the haptics in the correct position. For this to happen, the leading haptic of the IOL, that is the first haptic coming out of the injector nozzle, needs to exit correctly from the injector nozzle, and the rest of the optic body and further haptic need to exit in a controlled manner to position the IOL in the capsular bag.

Lenses whose leading haptic exits the injector in the wrong orientation are not acceptable for injection into the eye, as they may finish in the wrong position, which typically cannot be corrected in situ, and may result in reduced optical performance, particularly for aspheric, toric and multifocal IOLs.

Presently, certain types of IOLs cannot be injected into the capsular bag of a patient's eye via the nozzle of a conventional injector so as to emerge in the desired orientation with sufficient reliability. Modifications to the injector have been proposed to improve the reliability of injection, but this can be complex and costly. Therefore, there is a need for an improved design of IOL for use with injectors that can deliver the required reliability of injection performance.

### SUMMARY OF THE INVENTION

According to a first aspect of the present invention there is provided a deformable intraocular lens, IOL, comprising:
a deformable optic having an annular edge around its periphery;
at least two haptics, each having a proximal portion and a distal portion relative to the optic, wherein each haptic extends from a different position on the annular edge of the optic and can be compressed; and
at least one tab extending from the annular edge of the optic at a position different from the proximal portion of each of the haptics.

In this way, a deformable IOL is provided for use with an injector and whose folding characteristics are adjusted by the presence of one or more tabs at the annular edge of the optic, thereby controlling the speed and orientation of the IOL on injection, and ensuring greater reliability of desired injection.

Preferably, the IOL comprises a respective tab for each haptic, wherein each tab extends from the annular edge of the optic at a position different from the proximal portion of each of the haptics and from each other tab. In this way, each tab can be located and designed to cooperate with its respective haptic, in combination with other haptics and tabs, to give the desired performance on injection.

Preferably, each tab is located at a position on the annular edge that is closer to the proximal portion of its respective haptic than to that of another haptic. In combination with its respective haptic way, the tab can influence behaviour and orientation of the IOL on injection.

Preferably, each tab is located closer azimuthally to the distal portion of its respective haptic than to the proximal portion of its respective haptic. In this way, there is some separation between the tab and the haptic along the edge of the optic, but the distal portion of the haptic curves round to be closer azimuthally to the tab whilst radially separated. Typically, an IOL with two haptics will have a Z- or an S-configuration.

Although the tab may have a uniform radial extent along its circumferential extent, it will typically have an apex that is distal to the annular edge of the optic. Preferably, the apex is rounded. In some embodiments each tab has a truncated triangular shape.

Preferably, the maximum radial extent of at least one tab from the annular edge of the optic is less than the maximum radial extent of at least one haptic. In this way the tab does not dominate over the haptic.

In some embodiments the maximum radial extent of at least one haptic from the annular edge of the optic is at least the radius of the optic. In any event, the haptic must be suitably dimensioned relative to the optic to position it stably and centrally within a subject's eye.

Preferably, the optic is formed from a foldable material. This allows the optic to fold for insertion into a subject's eye by injection. The foldable material may be one of hydrophilic acrylic, hydrophobic acrylic, and silicone, although other materials are possible.

In some embodiments at least one haptic comprises an aperture. This requires less material and can alter the behaviour of the haptic. Likewise, in some embodiments at least one tab comprises an aperture. Each haptic and/or each tab may have more than one aperture.

In some preferred embodiments the IOL additionally comprises, around the optic, an annular rim that, in use, is in contact with the posterior capsular sac. This can help prevent epithelial cells from migrating to the optic region. The IOL may also additionally comprises, around the optic an annular rim that, in use, is on the anterior surface of the lens.

Preferably, the at least one tab is thinner than the annular edge of the optic. This ensures it does not interfere with a rim of the optic and its engagement with the capsular sac. The at least one tab may have the same thickness as at least one haptic, which can simplify fabrication.

As will be appreciated by those skilled in the art, the present invention provides an improved IOL which provides for injection into the capsular bag of a patient's eye via an injector nozzle so as to emerge in the desired positioning with good reliability. Further variations and embellishments will become apparent to the skilled person in light of this disclosure.

### BRIEF DESCRIPTION OF THE DRAWINGS

Examples of the present invention will now be described in detail with reference to the accompanying drawings, in which:

FIG. 1 is a plan view of an intraocular lens (IOL);

FIGS. 2A and 2B are respectively plan and cut-away side views of an IOL in which the thickness of the annular rim is greatest in the region of the optic binding the haptic;

FIG. 3A is a plan view of an intraocular lens according to the invention;

FIG. 3B is a plan view of the lens of FIG. 3A illustrating the radial extent of features by circular markers;

FIG. 4 is another plan view of the lens of FIG. 3A in a different orientation;

FIG. 5A shows the folding line for an intraocular lens with tabs according to the invention and FIG. 5B shows the folding line for an intraocular lens without tabs;

FIG. 6 shows six example intraocular lenses of the invention with tabs of different shape, size and position; and

FIGS. 7A, 7B, and 7C illustrate three IOL configurations with differing amounts of material connection between tab and haptic along the annular edge of the optic.

### DETAILED DESCRIPTION

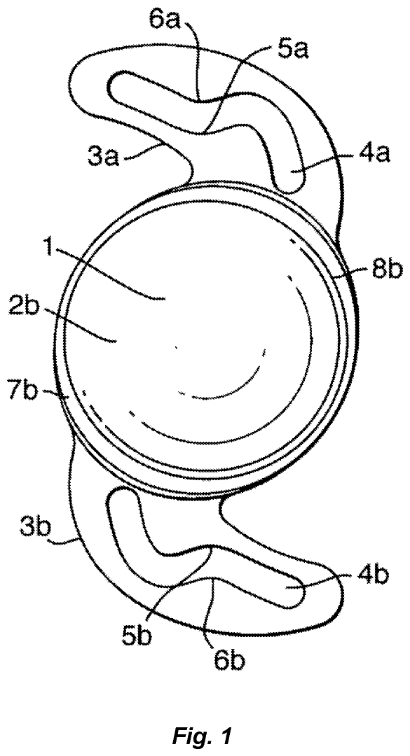

FIG. 1 illustrates an example of an intraocular lens (IOL) 10 having an optic 1, comprising convex faces (only one face, the posterior face 2b is shown) and two haptics 3a and 3b. Each haptic extends from the edge of the optic 1 and has a proximal portion and a distal portion. In other examples, the optic may have concave and/or convex faces.

As illustrated, the IOL is positioned with the two haptics in the ‘Z’ position, which is the preferred in situ orientation for the haptics when located in the capsular bag of the eye as seen by the surgeon implanting the IOL. This is as compared to a mirror reflection in which the haptics would face the opposite direction in an ‘S’ position.

Although not necessary, each haptic comprises an aperture, respectively 4a and 4b. Opposed points of each aperture, at 5a and 6a, and at 5b and 6b, are shown. These haptics are designed to be compressible for ease of insertion and to improve stability and centration of the IOL within the subject's eye.

The features of the haptics are such that initial compression of the haptic leads to abutment of opposite walls of the aperture, bringing the opposed points 5a and 6a, and 5b and 6b, into contact, thereby defining a proximal part that is fully compressed and a distal part that can undergo further compression. Such further compression brings the distal end of each haptic substantially into contact with the periphery of the optic, to give an essentially elliptical shape, in plan.

Furthermore, in this particular example, the lens comprises an annular rim 7b on the posterior face 2b of the optic 1, although this need not be present. The face 2b of the optic 1 can have various surface profiles according to the particular application. The periphery 8b of the posterior optic face 2b is also shown. The haptics, in conjunction with capsular shrinkage, hold the capsular sac tight against the posterior annular rim, such that epithelial cells are prevented from migrating to the optic region. This inhibits the onset of posterior capsular opacification (PCO).

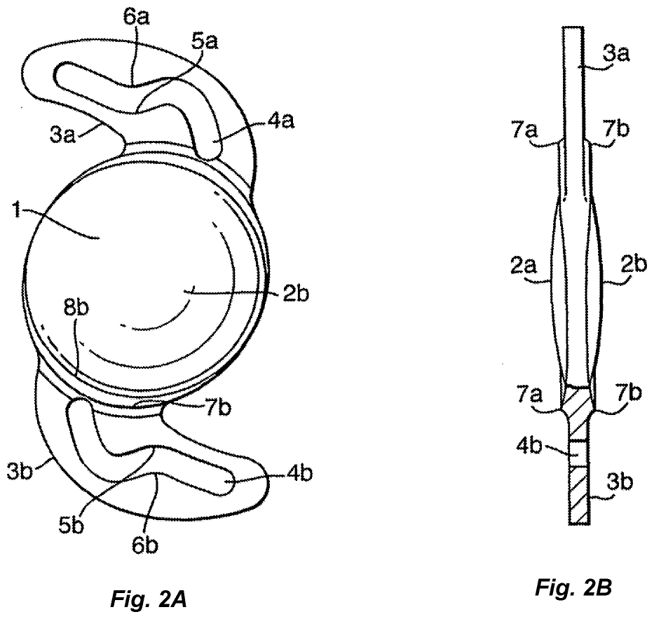

FIGS. 2A and 2B show another example of an IOL, each comprising a biconvex optic 1, having an anterior face 2a and a posterior face 2b. The lens comprises compressible haptics 3a and 3b, and annular rims 7a and 7b. In each case, the posterior capsular sac compresses the haptics, such that the posterior annular rim 7b is held tight against the posterior sac. The lens shown in FIGS. 2A and 2B is similar to that of FIG. 1 except in that the thickness of the annular rims is greatest in the region of the optic binding the haptic.

IOLs, such as described above, need to emerge into the capsular bag with the two haptics in the desired position when injected through an injector nozzle for MICS. Typically, this will be the Z-position, although in some applications it might be the S-position. The Z-position allows surgeons to rotate the lens clockwise for accurate rotational alignment in case of toricity, but also keeps the edge against the capsular bag, thereby protecting from posterior capsular opacification (PCO). For this process to happen, the leading haptic of the IOL, that is the first haptic coming out of the injector nozzle, needs to look left at its exit from the injector nozzle, and the rest of the optic body and second haptic need to exit in a controlled manner to position the IOL in the capsular bag.

Lenses designed to be implanted in the Z-orientation whose leading haptics exit the injector nozzle looking right are not acceptable for injection into the eye, as they will finish in the S-position, in which case they can later not be rotated from S to Z-position in the capsular bag, or the leading haptic will have to rotate itself backwards, which is also not possible because of the capsular bag will be against it, and therefore they will also finish in S position. This may result in reduced optical performance, particularly for aspheric, toric and multifocal IOLs.

With current hydrophilic lenses, when injected in to the eye of a patient, the lens haptics generally exit the nozzle of the injector in a satisfactory manner. However, it has been observed that some lenses can exit from the nozzle in a defective manner and inject incorrectly.

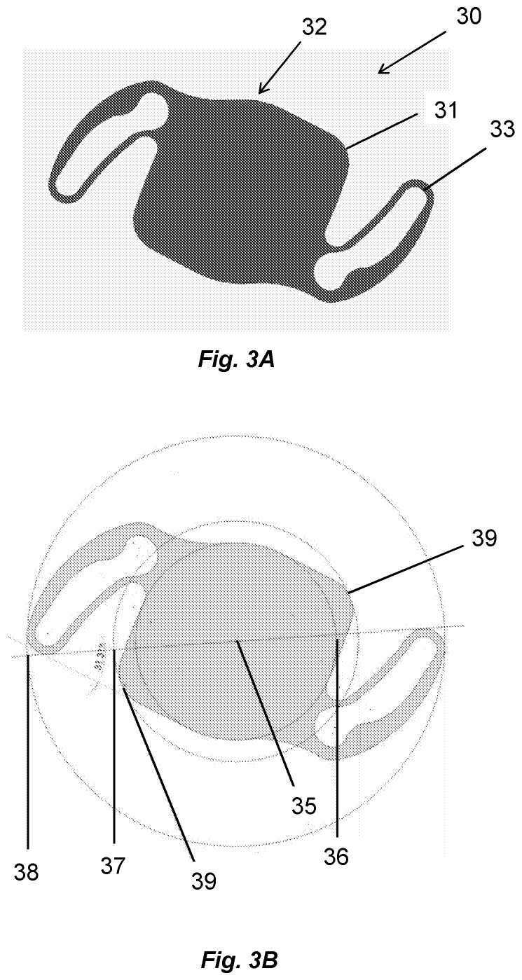

As shown in the image of FIG. 3A, this problem has been mitigated according to the invention by providing the IOL 30 with tabs 31 extending from the peripheral edge of the optic 32. It is noted that the tabs 31 are not located in the same peripheral position as the haptics 33. In this example the two haptics include apertures of the type illustrated in FIGS. 1 and 2A, whereas the tabs do not. However, the tabs may also include apertures, and conversely the haptics may not, according to the particular application.

FIG. 3B shows the IOL of FIG. 3B with the maximum spatial extent of certain features denoted by three circular markers 36, 37 and 38 centered on the centre 35 of the optic. As illustrated, the tabs extend from the otherwise generally circular periphery 36 of the IOL optic in a broadly triangular shape with rounded apex. In this case the diameter of the optic is 6 mm whilst the outer circle 38 defined by the maximum extent of the two haptics has a diameter of 12.5 mm. The apex 39 of each tab, which defines the inner circle 38, is located some distance from the edge of the optic, but not as far as the furthest point on each haptic measured from a tangent to the periphery of the optic at the point where the haptic joins the optic.

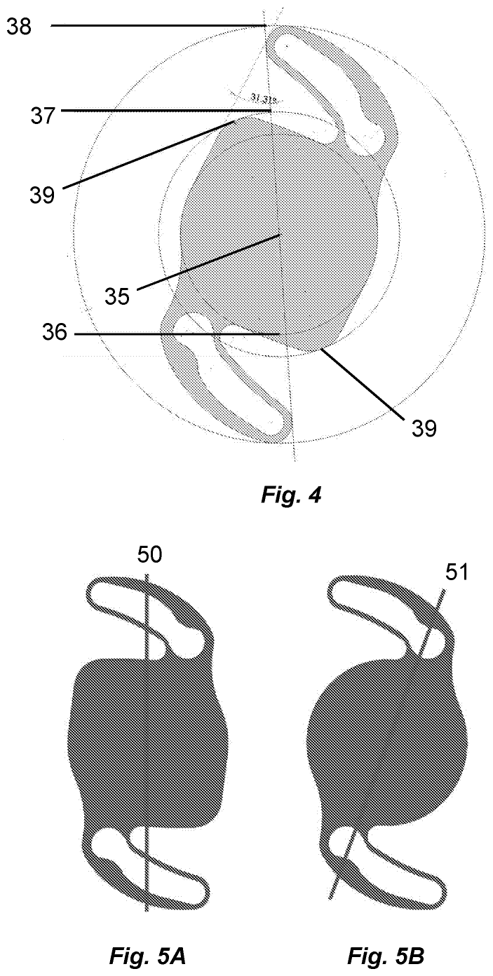

FIG. 4 illustrates the IOL of FIGS. 3A and 3B in a slightly different orientation. Again the maximum radial extent of the optic, the tabs, and the haptics is denoted by a respective circle 36, 37 and 38. As shown, the diameter of the circle 37 encompassing the two tabs lies between the diameter of the optic circle 36 and the haptic circle 38. In this particular example the ratio of the tab circle diameter to the optic diameter is about 1.2:1 and the ratio of the haptic circle diameter to the optic diameter is about 2:1.

As also indicated in FIG. 4, in this example a tangent along the slope of a tab towards the nearest haptic intersects a radial line from the centre of the optic at the point where the radial line touches the part of the haptic most azimuthally distant from where it adjoins the optic. Although the above examples illustrate an optic with a 6 mm diameter, the optic may have a different diameter, which will typically be in the range 3 to 11 mm.

The tabs shown in the examples of FIGS. 3A, 3B and 4 control both the haptic orientation on exit from an injector and also the speed (or IOL ‘shooting’) of the injection. Accordingly, the presence of this feature can significantly reduce the likelihood of defective MICS injections of an IOL of any power or material, as the IOL emerges in the capsular bag more reliably with the haptics in the Z-position.

FIGS. 5A and 5B illustrate, respectively, the line of folding of an IOL with tabs according to the invention and of a more conventional IOL without tabs. As can be seen, in FIG. 5B the line of folding 51 is essentially along a diametrical line between opposing points at which the haptics adjoin the optic. In FIG. 5A the line of folding 50 is moved to a diametrical line between opposing points located between the haptics and their respective nearest tab. Indeed, said points correspond fairly closely to where the tab first extends from the optic and slopes away from the nearest haptic. This modification to the line of folding caused by the presence of the tabs contributes to the different dynamics of the IOL during injection, which results in the IOL emerging in the right configuration.

The shape, position, dimension and number of tabs can be adjusted to optimise performance, depending on the precise design of the IOL. This includes the number and shape of the haptics, which can have any open or closed C-loop design or asymmetric design. Thus, there may be a corresponding tab for each haptic, or else there may be fewer or more, for example one, two three or four tabs. The tabs will typically be thinner than the central portion of the optic and have a constant thickness equivalent to the thickness of the haptic, although they may be thinner or thicker and/or tapered in thickness. In some embodiments the tab is thinner than the optic at its peripheral edge, thus ensuring that the raised edge integrity of the optic is not compromised.

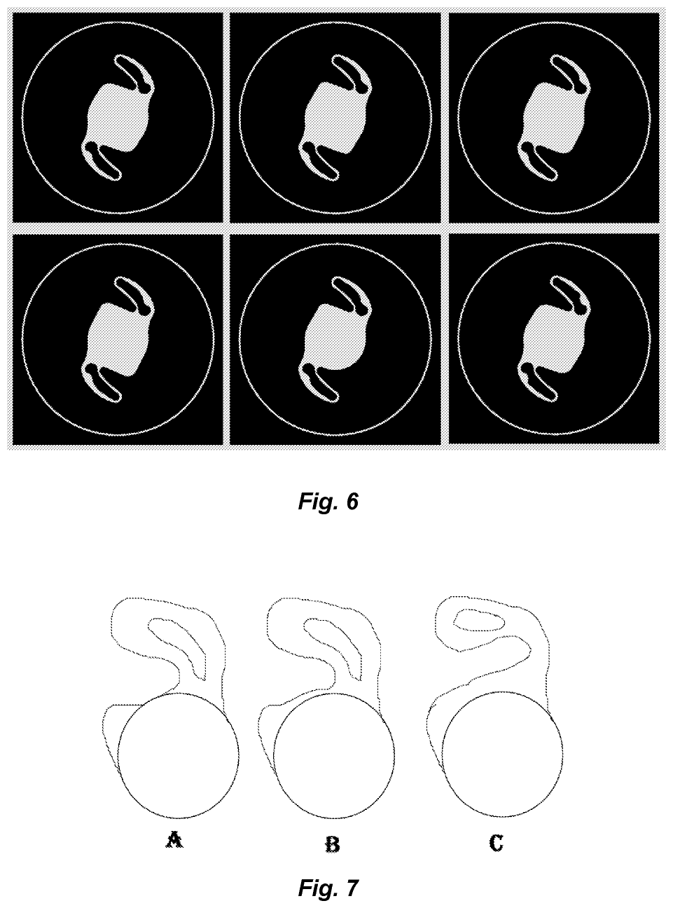

Injection using the same lens has been tested when varying slightly the size or shape of the tabs, and it has been found that that the right choice of symmetry between volume of haptic and tab at both sides of the lens optic body improves the orientation of the haptic at its exit from the injector nozzle. FIG. 6 illustrates several such examples.

In addition, the tab design has been tested for injection through a nozzle for lenses with different thicknesses, indicating that these may further benefit from an optimal haptic orientation after injection by modifying slightly the size and/or shape of the tabs. The tabs may include one or more apertures or indeed be substantially hollow to provide the desired characteristics.

The tabs may be completely distinct from the haptics or may connect in the region of the annular edge of the optic. FIG. 7 shows several such examples, with only a single haptic and tab illustrated. In FIG. 7A there is a distinct gap along the annular edge between the tab and the haptic, whereas in FIG. 7B there is a narrow strip of material connecting the tab and proximal region of the haptic along the annular edge. In FIG. 7C there is a more pronounced continuation of material along the annular edge connecting the tab and the proximal region of the haptic. In some embodiments the tab can be a deformed part of the annular edge, if it has been deformed for this purpose.

When under compression within the capsular bag, the haptic tips of an open or closed loop IOL are designed to fold inwards towards the optic body to counteract the forces applied by the eye and ensure centration and stability within a wide range of eye sizes. An additional potential benefit of the present invention is that, when under full compression, the tips can then engage with the edge of the tabs to create a more stable lens platform within the capsular bag. This is in a similar fashion to existing ‘plate’ haptic lenses that are designed with no compression ability, and therefore potential instability issues with larger or smaller eyes, but excellent in-bag stability for average capsular bag sizes.

Other potential benefits of the IOL design of the present invention may include reduced positive and/or negative dysphotopsia, improved manipulation within the eye during surgery, and reduced risk of the IOL exiting capsular bag via the anterior vault if irregular or torn capsularhexis is formed.

In summary, the present invention provides an innovative IOL with tabs that can take a range of different forms optimised to ensure reliable injection into the capsular bag of an eye with correct placement of the haptics, even for IOLs made of thicker and/or stiffer material. As will be appreciated by those skilled in the art, various modifications of the invention are possible based on the foregoing teaching.

1. A deformable intraocular lens (IOL), comprising:
a deformable optic having an annular edge around its periphery;
at least two haptics, each having a proximal portion and a distal portion relative to the optic, wherein each haptic extends from a different position on the annular edge of the optic and can be compressed; and
at least one tab extending from the annular edge of the optic at a position different from the proximal portion of each of the haptics.

2. The lens according to claim 1, wherein the IOL comprises a respective tab for each haptic, and wherein each tab extends from the annular edge of the optic at a position different from the proximal portion of each of the haptics and from each other tab.

3. The lens according to claim 2, wherein each tab is located at a position on the annular edge that is closer to the proximal portion of its respective haptic than to that of another haptic.

4. The lens according to claim 3, wherein each tab is located closer azimuthally to the distal portion of its respective haptic than to the proximal portion of its respective haptic

5. The lens according to claim 1, wherein each tab has an apex distal to the annular edge of the optic.

6. The lens according to claim 5, wherein the apex is rounded.

7. The lens according to claim 6, wherein each tab has a truncated triangular shape.

8. The lens according to claim 1, wherein the maximum radial extent of at least one tab from the annular edge of the optic is less than the maximum radial extent of at least one haptic.

9. The lens according to claim 1, wherein the maximum radial extent of at least one haptic from the annular edge of the optic is at least the radius of the optic

10. The lens according to claim 1, wherein the optic is formed from a foldable material.

11. The lens according to claim 10, wherein the foldable material is one of hydrophilic acrylic, hydrophobic acrylic, and silicone.

12. The lens according to claim 1, wherein at least one haptic comprises an aperture.

13. The lens according to claim 1, wherein at least one tab comprises an aperture.

14. The lens according to claim 1, wherein the IOL additionally comprises, around the optic, an annular rim that, in use, is in contact with the posterior capsular sac.

15. The lens according to claim 1, wherein the IOL additionally comprises, around the optic an annular rim that, in use, is on the anterior surface of the lens.

16. The lens according to claim 1, wherein the at least one tab is thinner than the annular edge of the optic.

17. The lens according to claim 1, wherein the at least one tab has the same thickness as at least one haptic.
